(19) United States
(12) Patent Application Publication
(10) Pub. No.: US 2020/0085706 A1
(43) Pub. Date: Mar. 19, 2020

(54) SKIN IMPROVING KIT AND FACE PACK

(71) Applicant: NIKKO SEISAKUSHO CO., LTD., Shizuoka (JP)

(72) Inventor: Miki MOCHIZUKI, Numazu-shi (JP)

(21) Appl. No.: 16/132,528

(22) Filed: Sep. 17, 2018

(51) Int. Cl. A61K 8/19; A61Q 19/00; A45D 44/00

(52) U.S. Cl. CPC A61K 8/19; A45D 2200/25; A45D 44/002; A61Q 19/00

(57) ABSTRACT

A skin improving kit includes a water retentive sheet; and an air-tightly sealed container that is filled with the hydrogen water. A face pack includes a water retentive sheet that has a face shape; and hydrogen water with which at least a surface of the water retentive sheet which comes into contact with a face has been impregnated.

### BACKGROUND

### Technical Field

The present invention relates to a skin improving kit and a face pack.

### Related Art

A method of drinking hydrogen water for maintaining health is drawing attention. Details of the mechanism thereof have not yet been elucidated, but it is thought that antioxidant effects of molecular hydrogen in hydrogen water contribute to the removal of active oxygen produced in a living body.

Products for enhancing cosmetic effects for the skin by using the antioxidant functions of hydrogen water are already available on the market Such products'already contain a hydrogen generating agent which generates hydrogen in a sheet or pad to be attached to the skin. When using the sheet or pad, moisture comes into contact with the hydrogen generating agent, and therefore hydrogen is generated (for example, refer to Japanese Unexamined Patent Application, First Publication No. 2015-167840).

### SUMMARY OF INVENTION

In products using a hydrogen generating agent, hydrogen is generated by a chemical reaction on the skin, leading to a burden on the skin.

A method of providing a commercially-available cotton puff or the like directly soaked with hydrogen water by a user, and then patting the skin with the provided puff or the like, has been conceived. However, hydrogen in hydrogen water is highly likely then to diffuse into the atmosphere.

An object of aspects of the present invention is to provide a skin improving kit and a face pack which are capable of allowing the effects of hydrogen water to be exhibited sufficiently.

The inventors of the present invention conducted intensive studies to obtain a skin improving effect by using hydrogen water. As a result, the inventors of the present invention have found that a desired effect will be obtained by using a kit in which hydrogen water with which an air-tightly sealed container is filled is combined with a water retentive sheet, and therefore have completed the invention of the present application.

Aspects of the present invention can adopt the following configurations.

(1) A skin improving kit including a water retentive sheet; hydrogen water; and an air-tightly sealed container that is filled with the hydrogen water.

(2) The skin improving kit according to (1), in which a hydrogen concentration of the hydrogen water is 0.6 ppm or more.

(3) The skin improving kit according to (1) or (2), further including a gas barrier sheet at covers one surface of the water retentive sheet.

(4) The skin improving kit according to (3), in which the gas barrier sheet is made of aluminum.

(5) The skin improving kit according to any one of (1) to (4), further including a container that contains a cosmetic liquid.

(6) The skin improving kit according to any one of (1) to (5), in which the water retentive sheet has a shape corresponding to a face

(7) The skin improving kit according to any one of) to (6), further including an air-tightly sealed container that has the water retentive sheet therein.

(8) A face pack including: a water retentive sheet that has a face shape; and hydrogen water with which at least a surface of the water retentive sheet which comes into contact with a face has been impregnated.

(9) The face pack according to (8), in which a hydrogen concentration of the hydrogen water is 0.6 ppm or more.

(10) The face pack according to (8) or (9), further including a gas barrier sheet on a surface of the water retentive sheet which does not come into contact with the face.

(11) The face pack according to (10), in which the gas barrier sheet is made of aluminum.

(12) The face, pack according to any one of (8) to (11), further including a cosmetic liquid with which at least a surface of the water retentive sheet which comes into contact with the face has been impregnated.

According to the aspects of the present invention, it is possible to effectively apply molecular hydrogen in hydrogen water to the skin, resulting in further enhanced cosmetic effects In particular, the effects thereof can be enhanced by covering the surface of the water retentive sheet which does not come in contact with the skin with the gas barrier sheet.

### BRIEF DESCRIPTION OF THE DRAWINGS

FIG. 1 is a diagram showing an example of a skin improving kit of the present embodiment,

FIG. 2 is a diagram showing an example of a method for using the skin improving kit of the present embodiment.

FIG. 3 is a diagram showing an example of the skin improving kit of the present embodiment.

FIG. 4 is a graph showing results of examples.

### DESCRIPTION OF EMBODIMENTS

Hereinafter, embodiments of the present invention will be explained, but he present invention is not limited thereto.

### Skin Improving Kit

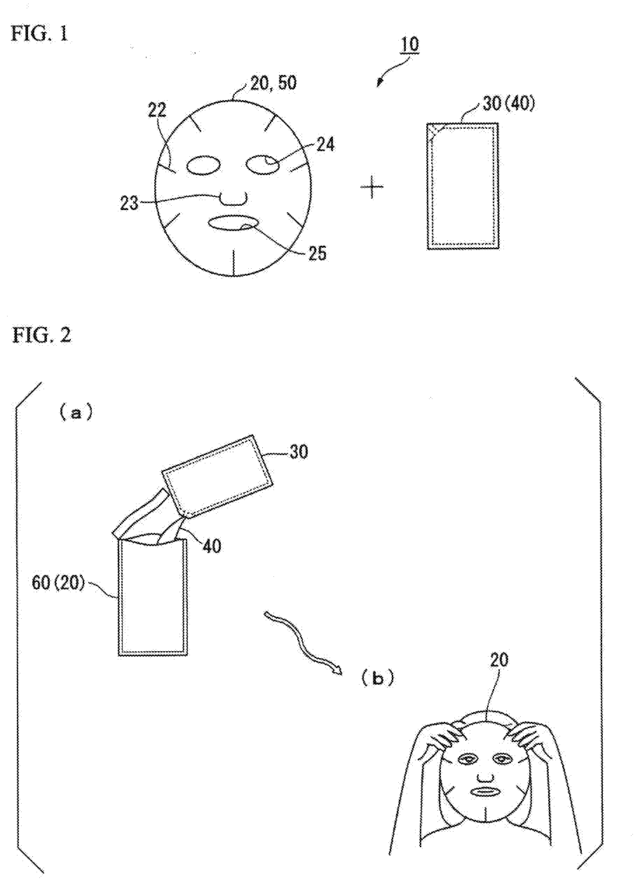

As shown in FIG. 1, in one embodiment, a skin improving kit 10 includes a water retentive sheet 20, a container (first container) 30, and a hydrogen water 40 with which the air-tightly sealed container 30 is filled.

the description hereinafter, the term “skin improvement” means, for example, a decrease in an amount of melanin pigments in the skin of a user, disappearance of erythema, an increase in a water content of the stratum corneum, a decrease in an amount of transepidermal water evaporation, an increase in skin viscoelasticity, and the like.

The water retentive sheet 20 is formed of a material and structure by which the sheet can be used in a state of coming into contact with the human skin, and has water absorption properties that enable impregnation of a predetermined amount of the hydrogen water 40. For example, the water retentive sheet 20 may have water-retention properties corresponding to a usage time of about 1, 5, 10, 15, 20, 25, 30, 40, 50, or 60 minutes, or longer. The above-described numerical values are representative examples, and in other examples, different numerical values can be applied.

The water retentive sheet 20 is preferably a sheet having properties not affecting the effect of hydrogen in hydrogen water. In one example, the water retentive sheet 20 is a nonwoven fabric mainly composed of cellulose fibers (regenerated cellulose fibers) such as supra, polynosic, lyocell, and rayon. The nonwoven fabric can be suitably applied to the water retentive sheet 20 from the viewpoint of not only the water absorption properties but also adhesiveness to the skin. In addition, examples of a material of the nonwoven fabric include nylon fibers, polyolefin fibers, cotton fibers, silk fibers, vinylon fibers, aramid fibers, and other fibers. In other examples, the water retentive sheet 20 can have a structure different from that of a nonwoven fabric. For example, the water retentive sheet 20 can have a fabric structure. In the water retentive sheet 20, one material may be used alone, or a plurality of materials may be used in combination.

The water retentive sheet 20 can have a single layer structure multilayer structure. The water retentive sheet 20 with a multilayer structure can have a plurality of layers having the same structure, and/or a plurality of layers having structures different from each other. For example, a multilayer structure is advantageous for water-retention properties of a high degree. In addition, at least one layer of the water retentive sheet 20 can have one surface which has been subjected to an embossing process.

The water retentive sheet 20 can have a shape corresponding to a part of the body to which the sheet is attached (the whole face, eyes, nasolabial folds, forehead, neckline, knee, elbow). For example, the water retentive sheet 20 can have a shape of a rectangle, circle, ellipse, and the like. In one example, the skin improving kit 10 is for the face, and the water retentive sheet 20 has a shape corresponding to the face. As will be described later, the skin improving kit 10 for the face is expected to have effects in beauty treatment.

As shown in FIG. 1, in a case where a part to which the water retentive sheet 20 is attached is the face, the water retentive sheet 20 has shape characteristics required for a face mask. Specifically, the water retentive sheet 20 has a shape where a pad is closely attached along curves of the face, Alternatively and/or additionally, the a retentive sheet 20 may have cuts 22 and 23 and/or openings 24 and 25, provided in parts corresponding to the eyes, nose, and mouth.

In one embodiment, the skin improving kit 10 further includes a gas barrier sheet 50 for covering one surface of the water retentive sheet 20 (a surface of the water retentive sheet 20 which does not come into contact with skin). The gas barrier sheet 50 has a lower hydrogen gas permeability than that of water retentive sheet 20. Examples of a material of the gas barrier sheet 50 include a plastic film such as polyethylene or a metal sheet such as aluminum.

The gas barrier sheet 50 can have a single layer structure or a multilayer structure. The gas barrier sheet 50 with a multilayer structure can have a plurality of layers of the same material, and/or a plurality of layers of materials different from each other.

In one example, the water retentive sheet 20 has a first surface on side coming into contact with the skin and a second surface on reverse side (on a side opposite) to the first surface. The gas barrier sheet 50 is disposed so as to cover the second surface of the water retentive sheet 20. In the water retentive sheet 20, the second surface can have a surface structure different front that of the first surface. Alternatively, in the water retentive sheet 20 with a multilayer structure, a layer on the first surface side can have a material and/or structure different from that of a layer on the second surface side. In one example, the water retentive sheet 20 has different levels of surface smoothness between the surface and the second surface. For example, compared to the first surface, the second surface may have a high level of smoothness. In the water retentive sheet 20, different surface structures between the first surface and the second surface are advantageous for enhancing the 2 or more functions of adhesiveness to the skin, the capacity to retain the hydrogen water 40, retention capacity of the gas barrier sheet 50, and the like.

In one example, the skin improving kit 10 can contain a sheet (gas barrier sheet 50) of a material having a lower hydrogen gas permeability than that of the water retentive sheet 20, as another constituent part separate from the water retentive sheet 20. In this case, at the time of use by a user, the sheet 50 is attached after the water retentive sheet 20 is attached. Alternatively, the user attaches the sheet 50 on one surface of the water retentive sheet 20 in advance, and then the user attaches the overlapped sheets 20 and 50 to the body. For example, on one surface of the water retentive sheet 20, marks, patterns, and/or color layers for distinguishing the front and back sides can be provided.

In other examples, the skin improving kit 10 includes the water retentive sheet 20 to which one surface of which the gas barrier sheet 50 has been attached in advance, the container 30, and the hydrogen water 40 with which the air-tightly sealed container 30 is filled. The gas barrier sheet 50 can have the shape same as or a different shape from that of the water retentive sheet 20. Similarly to the water retentive sheet 20, the gas barrier sheet 50 can have the cuts 22 and 23 and/or openings 24 and 25, provided in parts corresponding to the eyes, nose, and mouth. Alternatively and/or additionally, the gas barrier sheet 50 can have the cuts and/or openings in parts different from those of the water retentive sheet 20.

With the gas barrier sheet 50 having a lower hydrogen gas permeability than that of the water retentive sheet 20, release of the hydrogen water 40 absorbed in the water retentive sheet from the surface on side not coming into contact with the skin can be effectively reduced, and therefore the effect of the hydrogen water 40 can be focused on the side coming, into contact with the skin. In a case where the sheet 50 is a sheet made of aluminum, such a function is reliably exhibited. The sheet 50 preferably has in film shape. Accordingly, the gas barrier sheet 50 influencing the adhesiveness of the water retentive sheet 20 on the skin is avoided or mitigated.

In one embodiment, the skin improving kit 10 can further include an air-tightly sealed container (second container) 60 having the water retentive sheet 20. The water retentive sheet 20 is in the air-tightly sealed container 60 until the sheet is used, and therefore a sanitary state can be maintained until the sheet is applied to the skin. It is preferable that, when using the kit, the container 60 be also usable for allowing the water retentive sheet 20 to absorb the hydrogen water 40. Specifically, the container 60 is preferably a container having a form of a pouch in which the water retentive sheet 20 can be packed in a state of being appropriately folded. Furthermore, as shown in FIG. 2, the container 60 is more preferably a container in which a pack is opened, and the hydrogen water 40 is poured into the water retentive sheet 20 to soak the water retentive sheet 20 with the hydrogen water 40. For example, the container 60 has a structure capable of forming an opening for inserting the hydrogen water into the container 60 while maintaining a state where the water retentive sheet 20 is within the container 60.

In one embodiment, the skin improving kit 10 is configured such that the user uses the water retentive sheet 20 after soaking the sheet with the hydrogen water 40, Therefore, it is preferable that the hydrogen water 40 retains hydrogen in water before start of use at a dissolved hydrogen concentration at which sufficient effects at the time of use can be provided.

In order to obtain skin-beautifying effects and the like, the dissolved hydrogen concentration in the hydrogen water 40 is preferably 0.6 ppm or more, more preferably 1.0 ppm or more, even more preferably 1.5 ppm or more, and particularly preferably 2.0 ppm or more. An upper limit value thereof is not particularly limited but is preferably 7 ppm or less and more preferably 5 ppm or less. In one example, the dissolved hydrogen concentration in the hydrogen water can be about 0.6, 0.8, 1.0, 1.2, 1.4, 1.6, 1.8, 2.0. 2.2, 2.4, 2.6, 2.8, 3.0, 3.2, 3.4, 3.6, 3.8, 4.0, 4.2, 4.4, 4.6, 4.8, 5.0, 5.5 6.0, 6.5, or 7.0 ppm. The above-described numerical values are representative examples, and in other examples, different numerical values can be applied.

A method for producing the hydrogen water 40 is not particularly limited, and examples of the method include an electrolysis method, pressurized dissolution method, gas-liquid mixing nozzle method, micro-nano bubble method, gas-liquid separation hollow fiber membrane method, and the like.

It is preferable that the hydrogen water 40 used in the skin improving kit 10 has a high concentration of hydrogen in a production stage, and it is more preferable that a decrease in the dissolved hydrogen concentration be suppressed until use. As the air-tightly sealed container 30 that is filled with the hydrogen water 40, a pouch on which a metal foil which is a material through which hydrogen penetrates is laminated, and a metal can are preferable. Specifically, for example, as the container 30, aluminum pouches, steel cans, or aluminum cans are applied. Furthermore, in production steps, it is preferable that full filling be performed to fully fill the container with the hydrogen water, and that the hydrogen water with which the container has been fully filled in an airtight manner does not come into contact with a gas other than hydrogen, so that a headspace is not generated.

In the skin improving kit 10, an amount of the hydrogen water 40 stored in the container 30 is preferably an amount sufficient to soak the entire water retentive sheet 20. In one example, the amount of the hydrogen water can be about 10, 20, 30, 40, 50, 60, 70, 80, 90 ml, or more, For example, in a case of a cosmetic kit for the face (face mask), a volume of the hydrogen water 40 can be set to about 20 to 30 ml. The above-described numerical values are representative examples, and in other examples, different numerical values can be applied.

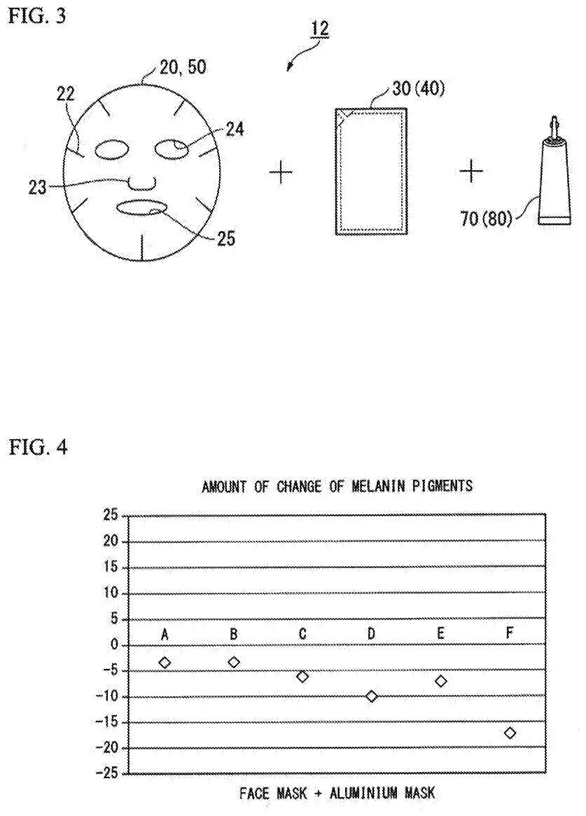

As shown in FIG. 3, in one embodiment, a skin improving kit 12 includes water retentive sheet 20, the container 30, the hydrogen water 40 with which the air-tightly sealed container 30 is filled, a container 70, and a cosmetic liquid 80 stored in the container 70.

In the skin improving kit 12, the cosmetic liquid 80 is not particularly limited as long as a liquid does not substantially reduce the effect of the hydrogen water 40. Examples of the cosmetic liquid 80 include various moisturizers, depigmenting agents, whitening agents, anti-wrinkle agents, and the like. As the cosmetic liquid 80, one kind thereof can be used, or a plurality of kinds thereof can be used. For example, a volume of the cosmetic liquid 80 is about several milliliters. In a case where cosmetic liquids 80 of a plurality of kinds are used, a liquid into which the respective cosmetic liquids have been mixed can be used, or each cosmetic liquid can be independently stored in separate containers (tube and the like). In a case where the water retentive sheet 20 is a face mask, when a plurality of kinds of the cosmetic liquids are stored in separate containers, it is possible to divide the cosmetic liquids into a cosmetic liquid to be applied to an entire pad, and a cosmetic liquid to be applied more to specific parts of the face (eyes, mouth, and the like).

In one embodiment, similarly to the skin improving kit 10, the skin improving kit 12 preferably includes the air-tightly scaled container 60 having the water retentive sheet 20 therein (refer to FIG. 2).

Using FIG. 2, an example of a method for using the skin improving kit 12 will be described.

First, the user opens the air-tightly sealed container 60 having the water retention sheet 20 therein, and pour e hydrogen water 40 and the cosmetic liquid into this container 60 so as to impregnate the water retention sheet 20. Next, a state in which the water retention sheet 20 that has been impregnated with the hydrogen water 40 and the cosmetic liquid is closely attached to the entire face of the user, is maintained for a predetermined time (for example, about 3 minutes). Next, the water retention sheet 20 is removed froth the face. This procedure is regularly performed, preferably 1 time or more every day, and more preferably twice a day in the morning and evening, and therefore the skin of the user can be improved.

In one embodiment, the skin improving kits 10 and 12 can be a product in which a package in which each of the above-described constituent parts is packed together for one procedure is provided, and a plurality of packages are packed together. Examples of the package include a disposable plastic container and the like.

According to the above-described skin improving kits 10 and 12 according to each embodiment, it is possible to effectively apply molecular hydrogen in the hydrogen water 40 to the skin, and thus the skin-beautifying effect can be obtained.

### Face Pack

In one embodiment, the face pack includes the water retentive sheet 20 that has a face shape; and the hydrogen water 40 with which at least a surface of the water retentive sheet 20 which comes into contact with the face has been impregnated. That is, the face pack is a pack of which application is limited to the face in the above-described skin improving kits 10 and 12, and therefore the description thereof will be omitted.

According to the face pack of the present embodiment, it is possible to effectively apply molecular hydrogen in the hydrogen mater 40 to the skin, and thus the effect of beauty treatment can be obtained.

### Example 1

### Face Mask Production Examples

The following masks were produced as sheets having cuts or openings in parts corresponding to the eyes, nose, mouth at the time of wearing the facemask, and the overall shapes thereof were circular.

(1) Face mask formed of a water absorbent sheet having water-retention properties

(2) Mask made of aluminum to be attached on a surface of the face mask of (1) above which does not come into contact with the face

### Example 2

### Examples of Using Face Masks

The face mask produced in Example 1 immersed in about 30 mL of hydrogen water (hydrogen concentration: 0.6 ppm) manufactured by K. K. NIKKO Corporation was used by 6 panelists continuously for 1 week twice a day in the morning and evening. Skin conditions before using the face mask and after using the face mask for 1 week were checked. This test was performed in a case where only the face mask of (1) above was attached, and in a case where the face mask of (1) and the aluminum mask of (2) above were attached. Checking of the skin conditions was performed by measuring a level of melanin pigments at a specific part using a Mexameter.

### Results

Among 6 panelists who used only the face mask impregnated with the hydrogen water, a decrease in the amount of the melanin pigments was confirmed in 2 panelists. Furthermore, FIG. 4 shows test results in a case where the aluminum mask was attached on the face mask impregnated with the hydrogen water. In FIG. 4, a vertical axis shows differences between the level of the melanin pigments after use and the level of the melanin pigments before use. As can be understood based on the results of FIG. 4, in the case where the aluminum mask was attached on the face mask, a decrease the amount of the melanin pigments could be confirmed in all panelists A to F as compared with before use.

What is claimed is:

1. A skin improving kit, comprising:
a water retentive sheet;
hydrogen water; and
an air-tightly sealed container that is filled with the hydrogen water.

2. The skin improving kit according to claim 1, wherein a hydrogen concentration of the hydrogen water is 0.6 ppm or more.

3. The skin improving kit according to claim 1 further comprising:
a gas barrier sheet that covers one surface of the water retentive sheet.

4. The skin improving kit according to claim 3, wherein the gas barrier sheet is made of aluminum.

5. The skin improving kit according to claim 1, further comprising:
a container that contains cosmetic liquid.

6. The skin improving kit according to claim 1, wherein the water retentive sheet has a shape corresponding to a face.

7. The skin improving kit according to claim 1 further comprising:
an air-tightly sealed container that has the water retentive sheet therein.

8. A face pack comprising:
a water retentive sheet that has a face shape; and
hydrogen water with which at least a surface of the water retentive sheet which comes into contact with a face has been impregnated.

9. The face pack according to claim 8, wherein a hydrogen concentration of the hydrogen water is 0.6 ppm or more.

10. The face pack according to claim 8, further comprising:
a gas barrier sheet on a surface of the water retentive sheet which does not come into contact with the face.

11. The face pack according to claim 10, wherein the gas barrier sheet is made of aluminum.

12. The face pack according to claim 8, further comprising:
a cosmetic liquid with which at least a surface of the water retentive sheet which comes into contact with the face has been impregnated.
